(19) United States
(12) Patent Application Publication
(10) Pub. No.: US 2009/0170801 A1
(43) Pub. Date: Jul. 2, 2009

(54) METHODS OF TREATMENT OF CARDIOVASCULAR AND CEREBROVASCULAR DISEASES WITH FUCOIDAN

(75) Inventor: Shouzhu HAO, Beijing (CN)

Correspondence Address: MATTHIAS SCHOLL, 14781 MEMORIAL DRIVE, SUITE 1319, HOUSTON, TX, 77079, US

(73) Assignee: Beijing Century Biocom Pharmaceutical Technology Co. Ltd., Beijing (CN)

(21) Appl. No.: 12/397,443

(22) Filed: Mar. 4, 2009

Related U.S. Application Data
(63) Continuation of application No. PCT/CN2007/002619, filed on Aug. 31, 2007.

(30) Foreign Application Priority Data
Sep. 4, 2006 (CN) 200610112391.1
Dec. 8, 2006 (CN) 200610140394.6

(51) Int. Cl. A61K 31/715; A61P 9/00

(52) U.S. Cl. 514/44

(57) ABSTRACT

A method for treating an ischemic cardiovascular or cerebrovascular disease comprising administrating to a patient in the need of such treatment a pharmaceutical composition comprising fucoidan.

### CROSS-REFERENCE TO RELATED APPLICATIONS

This application is a continuation of International Patent Application No. PCT/CN2007/002619 with an international filing date of Aug. 31, 2007, designating the United States, now pending, and further claims priority benefits to Chinese Patent Application No. 200610112391.1 filed Sep. 4, 2006 and to Chinese Patent Application No. 200610140394.6 filed Dec. 8, 2006. The contents of all of the aforementioned applications, including any intervening amendments thereto, are incorporated herein by reference.

### BACKGROUND OF THE INVENTION

1. Field of the Invention

This invention relates to methods of treating cardiovascular and/or cerebrovascular diseases with fucoidan.

2. Description of the Related Art

Fucoidans are a class of sulfated polysaccharides found mainly in various species of brown seaweed. Fucoidans were first isolated in 1913 from Laminaria digitata (oarweed) by Kylin who initially named them fucoidin because of L-fucose found in the acid hydrolyzate of the seaweed. Subsequently, this class of polysaccharides began to be referred to as fucoidans following standard IUPAC nomenclature. Nevertheless, other names for this class of polysaccharides are also in use including fucan, sulfated fucan, fucosan, fucosan sulfuric ester, fucus polysaccharide, fucose polysaccharide, brown algae syrup, or brown algae polysaccharide sulfuric ester.

The chemical makeup of many fucoidans has since been fully elucidated. Fucoidans have complex chemical structure, mainly comprising fucose and sulfate groups, and additionally often also comprising various groups derived from other compounds, such as galactose, xylose, uronic acid, depending on which algae the fucoidans are isolated from. For example, fucoidan from kelp is composed of different monosaccharides, such as fucose, galactose, xylose, glucuronic acid, arabinose, and so on, and particularly fucose and galactose being present in the weight ratio of about 3:1.

The chemical structure of fucoidans is complex, and varies greatly in different algae. Up to now, the structure of fucoidans extracted from Fucus vesiculosus and Ascophyllum nodosum has been most studied. The fucoidan from Fucus vesiculosus is mainly linked by α(1→3) glycosidic bonds, and the sulfation mainly occurs at the C2 and C3 positions. The fucoidan from Ascophyllum nodosum contains a large number of α(1→3) and α(1→4) glycosidic bonds.

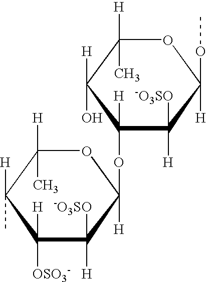

### Repeat Unit of Fucoidan Isolated from Fucus vesiculosus and Ascophyllum nodosum

The structure of fucoidan from other brown algae has also reported. For example, the fucoidan from Ecklonia kurome is mainly linked by α(1→3) glycosidic bonds, and sulfation occurs at the C₄ position. The main chain of fucoidan from Cladosiphon okamuranus and Chorda filum comprises fucose linked by α(1→3) glycosidic bonds, and sulfation occurs at the C4 position; furthermore, the fucoidans of the two species comprise a few of 2-O-acetyls groups.

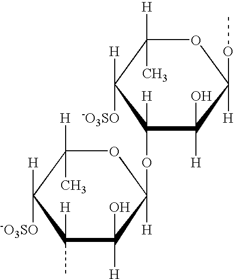

### Repeat Unit of Fucoidan Isolated from Ecklonia kurome

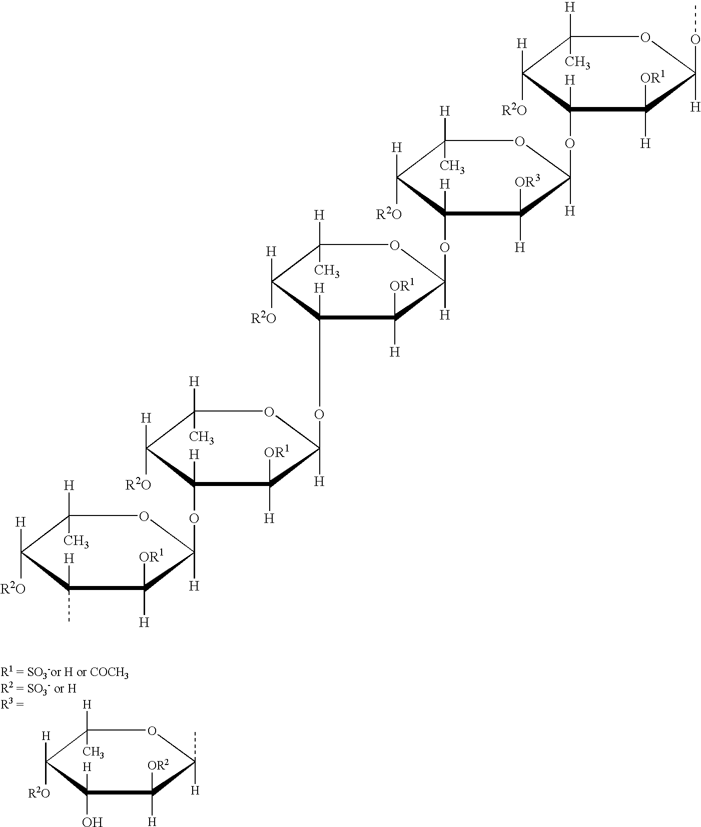

### Repeat Unit of Fucoidan Isolated from Chorda filum

It has been shown that the fucoidan from kelp is mainly composed of L-fucose linked by α(1→3) glycosidic bonds, and sulfation occurs at C2 or C4. Some contend, however, that there are also side chains in the fucoidan from kelp composed of L-fucose linked by (112) glycosidic bonds. This structure would be is similar to the structure of fucoidan from Chorda filum shown above with the exception that there are also acetyl groups in Chorda filum, and the percentage of substituted groups is different between the two species. Furthermore, the fucoidan from kelp comprises monosaccharides, such as galactose, xylose, and rhamnose. Galactose may be involved in constituting the main chain, while the xylose and rhamnose may be involved in constituting the side chain.

Preparation methods and medical application of fucoidans have been disclosed in literature. For example, Jap. Patent No. 46-2248 discloses that reacting cetyl pyridine chloride or cetyltrimethylammonium bromide with fucoidan yields a quaternary ammonium salt complex. According to the solubility difference of the complex in salt, algin, a neutral polysaccharide and other impurities are removed by purification with ethyl alcohol, methyl alcohol and ion exchange resin, and the purified fucoidan is obtained.

CN1129109A discloses an alkali agglutination separation method comprising soaking air-dried kelp, filtering several times, extracting with alcohol twice, washing with alcohol once, regulating the pH range and so on.

CN1344565A discloses a method comprising pre-treating raw materials, stirring and extracting under a certain temperature, centrifugating, concentrating, precipitating with alcohol, dehydrating with anhydrous alcohol, and so on.

CN1560086A discloses a method of preparation of fucoidan having high content of sulfate group, comprising extracting brown algae with hot water or acid water to obtain an extract containing fucoidan, concentrating the extract to the weight percentage of polysaccharide between 2% and 10%, regulating the pH value to between 5 and 8, adding chitosan solution and stirring, centrifuging or filtering to collect deposit, extracting the deposit 2-4 times with 5-10 times the weight of salt solution, centrifuging or filtering to collect a clear solution; desalting the clear solution by dialyzing or ultra filtering.

Additionally, CN1670028A, CN1392160A and CN1197674A each disclose a flocculation method of preparing algal polysaccharide.

CN1547478A discloses a use of fucoidan in treating adhesion, arthritis and psorlasis.

Furthermore, the above-mentioned references further disclose that fucoidan has one or more of the following properties: anticoagulative, immunity enhancing, anti-tumoral, anti-viral, decreasing blood glucose, radiation-protective, ascite-suppressing, and so on.

Up to now, use of fucoidan in treating coronary heart disease and stroke has not been disclosed.

### BRIEF SUMMARY OF THE INVENTION

Therefore, it is one objective of the invention to provide a method for the treatment of ischemic cardiovascular and cerebrovascular diseases.

Specifically, in one embodiment of the invention, provided is a method for the treatment of ischemic cardiovascular or cerebrovascular disease comprising administrating to a patient in need thereof a pharmaceutical compositions comprising fucoidan. The ischemic cardiovascular and cerebrovascular diseases include but are not limited to coronary heart disease and stroke. The coronary heart disease includes but is not limited to symptomless coronary heart disease, angina, cardiac infarction, arrhythmia, and sudden death. The stroke includes but is not limited to cerebral hemorrhage and cerebral infarction.

In certain classes of this embodiment, the fucoidan is extracted from cultivated kelp, or from wild brown algae such as gulfweed, Undaria pinnatifida, Sargassum fusiform, Sargassum thunbergii, Sargasnam kjellmanianum, Ecklonia kurome, Fucus vesiculosus, and Ascophyllum nodosum, etc. In particular, the fucoidan used in the methods of this invention is extracted from kelp.

In certain classes of this embodiment, the molecular weight of fucoidan is between 10 kDa and 1000 kDa, particularly between 50 kDa and 800 kDa, or 100 kDa and 700 kDa, more particularly between 150 kDa and 500 kDa, and most particularly between 200 kDa and 400 kDa.

In another embodiment of the invention, provided is a pharmaceutical composition comprising fucoidan. The pharmaceutical composition comprises an effective dose of fucoidan and at least one pharmaceutically acceptable excipient.

The mode of administration of the pharmaceutical composition includes but is not limited to intravenous injection, intramuscular injection, hypodermic injection, topical application, oral administration, and rectal administration.

The dosage form of the pharmaceutical composition includes but is not limited to parenteral solution, lyophilized injectable powder, injection microspheres, liposomes, tablets, capsules, water agent, powder, cataplasma, sprayable solution, granular formulation, soft capsules, drop pills, gel, patch, paste, etc. A parenteral solution, lyophilized injectable powder, tablets, and capsules are preferable. Appropriate dosage form is easily prepared by those skilled in the art according to the prior art and common sense.

In certain classes of this embodiment, the weight percentage of fucoidan with respect to the pharmaceutical composition is ≧50%, particularly ≧70%, more particularly ≧90%, and the most particularly ≧95%. The fucoidan content in a unit-dose is between 1 mg and 1000 mg, particularly between 10 mg and 800 mg, more particularly between 30 mg and 500 mg, or between 30 mg and 300 mg, and most particularly between 50 mg and 100 mg.

In certain classes of this embodiment, fucoidan, and particularly fucoidan extracted from kelp, decreases the degree and scope of myocardial infarction, and reduces the extent of myocardial infarction. In this application, the molecular weight of fucoidan is particularly between 200 kDa and 400 kDa.

In certain classes of this embodiment, fucoidan, and particularly fucoidan extracted from kelp, decreases ischemia reperfusion-induced brain edema, reduces intracranial pressure, and improves brain microcirculation, so that the production of superoxide dismutase is increased, and meanwhile the vitality of lactate dehydrogenase is reduced. In this application, the molecular weight of fucoidan is preferably between 200 kDa and 400 kDa.

The fucoidan used in methods of the present invention was extracted, purified and graded according to the following methods.

1. Extracting

Fucoidan was extracted with water, diluted acid, or calcium chloride solution, then lead hydroxide, aluminum hydroxide, ethanol, or quaternary ammonium salts were added as cationic surfactants to the extract, to allow fucoidan to precipitate out. In order to reduce the rate of dissolution of pigment and proteins, algae can be pre-treated with a high concentration of alcohol or formaldehyde solution prior to extraction. Techniques such as microwave extraction, ultrasonic extraction, and flocculation polymer precipitation extraction can be used.

2. Purifying

Ethanol Re-Precipitation Method

Crude fucoidan aqueous solution was extracted with hot water, and 20% ethanol was added in the presence of 0.05M MgCl₂ to remove impurities such as water-soluble algin (Nishide Eiichi, Bulletin of the Japanese Society of Scientific Fisheries, 1982, 48(12):1771).

Crude fucoidan extracted from Sargassum horneri (turn) was dissolved in water, 4M CaCl₂ and 30% ethanol were added successively to remove algin, then 80% ethanol was added and purified fucoidan precipitated out (Wang Zuoyun, Zhao Xuewu, Isolation and purification of fucoidan, laminaran and algin from Sargassum horneri (turn), Journal of Fisheries of China, 1985, 9(1):71).

Quaternary Ammonium Salts Precipitation Method

Fucoidan precipitated out by reacting cationic surfactants such as cetyl pyridine chloride (CPC) or cetyltrimethylammonium bromide (CTAB) with a polymer electrolyte.

In the extraction and purification process, a dialysis method is generally used for the removal of ions and small molecules. An ultrafiltration separation method is also used to exclude substances with smaller molecular weights. Enzymatic digestion is sometimes used to remove laminaran and proteins which are intermixed in an extract solution.

Glucanase and alcalase can be used for the removal of laminaran and proteins during extraction and purification process (Fleury N and Lahaye M; Studies on by-products from the industrial extraction of alginate 2. Chemical structure analysis of fucans from the leach-water. J Appl Phycol, 1993, 5: 605-610). Additionally, since laminaran is electrically neutral and fucoidan is generally in the form of polyanions, ion exchange resin method can be used to separate the two compounds.

3. Fractionation

Fucoidan has a complex chemical structure which makes chromatographic and electrophoretic fractionation of crude fucoidan mixtures feasible. A conventional fractionation method involves ethanol precipitation, i.e., a stepwise increasing concentration of ethanol is used to precipitate out different fractions.

Another method involves chromatographic fractionation, e.g., gel filtration chromatography or ion exchange chromatography. Ion-exchange chromatography separates polysaccharides into fractions having different electric charge, and gel filtration chromatography separates polysaccharides according to molecular weight.

Additionally, an ultrafiltration membrane of a certain molecular weight rating can be used to fractionate fucoidans so as to obtain fractions having a certain molecular weight.

### DETAILED DESCRIPTION OF THE EMBODIMENTS

Detailed description will be given below with reference to accompanying examples. The examples are provided herein to just describe the present invention. It will be obvious to those skilled in the art that changes and modifications may be made without departing from the invention in its broader aspects, and therefore, the aim in the appended claims is to cover all such changes and modifications as fall within the true spirit and scope of the invention.

### Example 1

### Preparation of Fucoidan

Seaweed was crushed, soaked in 3.7% formaldehyde solution overnight, and then distilled water was added. The mixture was boiled to yield an extract. The extract was filtered through diatomite. The filtrate was firstly dialyzed for a day with running tap water, and then dialyzed for another day with distilled water.

The dialysate was concentrated, and ethanol was added dropwise (until the concentration of ethanol was up to 75%) to obtain a precipitate. The precipitate was dried to give crude fucoidan. The crude product was re-dissolved in water. 20% ethanol was added in the presence of 0.05 M MgCl₂ to precipitate and remove water-soluble algin. The filtrate was dialysed, concentrated, and precipitated with 75% ethanol. The precipitate was dried to give purified fucoidan.

The molecular weight of the purified fucoidan was measured by high-performance gel permeation chromatography.

Following the above-mentioned method, fucoidans from four kinds of seaweeds, namely, Sargassum kjellmanianum, Sargassum thunbergii, Sargassum ilicifolium, and kelp were separately prepared.

The chemical composition of the obtained fucoidans is listed below:

| | | | Peak | | | | | |
|---|---|---|---|---|---|---|---|---|
| | | | molecular | | | | | |
| | Fucose | SO₄²⁻ | weight | Ash | Molar ratio of monosaccharide | | | |
| Seaweed | (%) | (%) | (kDa) | (%) | Fucose | Galactose | Xylose | Glucose |
| Sargassum | 26.5 | 14.8 | 980 | 20.8 | 1.00 | 0.24 | 0.05 | 0.04 |
| kjellmanianum | | | | | | | | |
| Sargassum | 25.4 | 17.0 | 650 | 22.6 | 1.00 | 0.24 | | 0.03 |
| thunbergii | | | | | | | | |
| Sargassum | 13.3 | 12.5 | 588 | 20.8 | 1.00 | 0.35 | 0.16 | 0.08 |
| ilicifolium | | | | | | | | |
| Kelp | 28.8 | 30.2 | 220 | 31.2 | 1.00 | 0.36 | | |

Preparation of Fucoidan Having Various Molecular Weights

The above-mentioned fucoidan from kelp was dissolved in water. Subsequently, the fucoidan was fractionated according to molecular weight using Pall Minimate small tangential flow ultrafiltration system, separately passing through ultra-filtration membranes having a molecular weight cut-off of 400 kDa, 200 kDa, 100 kDa, and 10 kDa. Three kinds of polysaccharide having molecular weight of between 200 kDa and 400 kDa, between 100 kDa and 200 kDa, and between 10 kDa and 100 kDa were obtained. The molecular weight was measured by high-performance gel permeation chromatography (HPGPC). The chemical compositions are listed below:

| | | | Peak | | | |
|---|---|---|---|---|---|---|
| | | | molecular | | Molar ratio of | |
| Fucoidan | Fucose | SO₄²⁻ | weight | Ash | monosaccharides | |
| (kDa) | (%) | (%) | (kDa) | (%) | Fucose | Galactose |
| 200-400 | 29.5 | 31.5 | 288 | 33.8 | 1.00 | 0.33 |
| 100-200 | 28.4 | 30.1 | 147 | 32.6 | 1.00 | 0.38 |
| 10-100 | 28.8 | 29.7 | 79 | 30.8 | 1.00 | 0.42 |

### Example 2

### Preparation of Fucoidan for Injection

500 mL of water for injection and 50 g of mannitol were added to 50 g of fucoidan extracted from kelp, the pH value being adjusted to 7.0, and the solution was freeze-dried, and packaged.

### Example 3

### Preparation of Fucoidan Tablets

Microcrystalline cellulose and polyvinylpyrrolidone were added to 50 g of fucoidan extracted from kelp. After mixing, an appropriate amount of water was added, and the ingredients were mixed, granulated and dried. Crosslinked sodium carboxymethyl cellulose, and magnesium stearate were added to the granules, mixed, and tableted. Each tablet contained 100 mg of fucoidan.

### Example 4

### Protecting Action of Fucoidan Against Myocardial Ischemia

Effect of Fucoidan on Hemodynamics and Myocardial Oxygen Consumption in Anesthetized Open-Chest Dogs

Healthy adult dogs (between 12 kg and 20 kg in body mass, male and female) were randomly divided into groups each group having 6 dogs. The control group was administrated equal volume of 0.9% normal saline. The positive group was administrated Ginkgo Biloba extract (4 mg/kg). The experiment group had two dosage groups, which was respectively administrated 4 mg/kg, and 16 mg/kg of fucoidan (molecular weight between 200 kDa and 400 kDa) from kelp by intravenous injection.

The dogs were anesthetized with i.v. sodium pentobarbital (30 mg/kg), administered to the back. The neck skin was cut, endotracheal intubation performed to connect an electric respirator. The right carotid artery was exposed and connected to an AP. 601G amplifier, and the blood pressure was measured. The femoral artery was exposed, connected to an AP. 601G amplifier. Ventricular cannulation was performed to measure left ventricular pressure and end diastolic pressure, and ±dp/dt max were measured with an electronic differentiator EQ-601G Thoracotomy was performed in the left fourth intercostals, the heart exposed, the pericardium excised, and cardiac surgery performed. The left circumflex coronary artery and aortic root were exposed, and an electromagnetic flowmeter probe was placed to measure coronary blood flow and aortic flow. Limbs were connected to perform limb lead and the standard II lead ECG was measured, and heart rate calculated. Femoral vein was exposed, and venous cannulation was performed for drug delivery. The above-mentioned indexes were simultaneously recorded in a polygraph.

After surgery and 15 minutes' of stability, the indexes were recorded before administration and at 3, 5, 10, 15, 20, 30, 45, 60, 90, 120, 150, 180 and 240 min after administration. Arterial blood and coronary sinus blood were collected before administration and at 45, 60, 90, 120, 180 and 240 min after administration. Blood oxygen content was measured using an oximeter (Kangni-158, US). The following secondary indexes were calculated according to applicable formulas: mean arterial pressure, cardiac index, stroke index, left ventricular stroke work index, total peripheral resistance, coronary resistance, myocardial oxygen consumption, myocardial oxygen consumption index, myocardial oxygen extraction ratio, myocardial blood flow, etc. The measured experimental data and percent change were compared with those of the control group, and t-test between groups was performed for statistical analysis.

Effect of Fucoidan on Dogs with Experimental Myocardial Infarction

Healthy adult dogs (the same as above) were randomly divided into groups with each group 6 dogs. The dogs were i.v. anesthetized with sodium pentobarbital (30 mg/kg), fixed in the back. The neck skin was cut, and endotracheal intubation was performed to connect an SC-3 artificial respirator. The lower one third of left anterior descending artery was exposed for ligation to cause myocardial infarction. A wet-type multi-point adsorption method was used to map EECG Provided were 32 mapping points comprising normal area (control points), infarct marginal area and the central area of infarction. After surgery the dogs were stabilized for 15 minutes. Meanwhile, femoral vein blood was collected and myocardium tris enzyme (AST, CPK, LDH) value was measured before administration. After the coronary artery was ligated for 15 minutes, the ST segment was significantly increased, which suggested that a model was established. Through femoral intravenous injection, the control group was administrated equal volume of 0.9% normal saline. The positive group was administrated Ginkgo Biloba extract (4 mg/kg). The experimental group was divided into two dose groups, which was respectively administrated 4 mg/kg, 16 mg/kg of fucoidan (molecular weight between 200 kDa and 400 kDa). EECG was recorded under normal conditions, after ligation, and at 3, 5, 10, 15, 20, 30, 45, 60, 90, 120, 150, 180, 240, 300, 360 min after administration. Σ-ST was expressed as the total increased mV number of the ST-segment, and N-ST was expressed as increased ST-segment lead number>2 mV. At 360 min after administration, blood was collected again to measure myocardium tris enzyme. After experiment, the heart was harvested and the total weight measured. The root of great vessel and atrial were cut along coronary sulcus to obtain the weight of left ventricle. The left ventricle was cut into 5 or 6 pieces cross-sectionally and equably. The pieces were stained with nitro blue tetrazolium (N-BT) for 15 min at constant temperature in a water bath at 37° C. The infarcted area was not colored, while the non-infarcted area was colored blue by NBT. The non-infarcted cardiac muscle which had been colored was cut, and the infarcted cardiac muscle which had not been colored was weighted. The weight was divided by the total heart weight and the ventricular weight respectively to obtain the percentage of the infarcted area in the total heart weight and in the ventricular weight. All experimental data was expressed as X̅±S, and t test was used to determine the significance of difference of mean value between groups.

Results

In the dosage group (16 mg/kg), the measured value of the effect of kelp fucoidan on the ischemia degree in dogs between 10 min and 240 min after administration is significantly different from that of the control group, and the change rate exhibits a significant inhibitory effect.

In the dosage group (16 mg/kg), the measured value of the effect of kelp fucoidan on the ischemia range in dogs between 10 min and 240 min after administration is significantly different from that of the control group, and the corresponding change rate exhibits a significant inhibitory effect between 10 min and 45 min.

Detailed results are shown in Tables 1, 2 and 3.

TABLE 1
Effect of kelp fucoidan on the degree of ischemia in dogs with myocardial
infarction (Σ-ST, Mv) (X ± s, n = 6)

| | Dosage | After administration (min) | | | |
|---|---|---|---|---|---|
| Groups | (mg/kg) | Ligation | 3 | 5 | 10 |
| Control | — | 186.00 ± 63.60 | 182.50 ± 47.62 | 167.33 ± 40.71 | 186.50 ± 45.72 |
| group | | | | | |
| % | | | 6.44 ± 43.27 | −2.43 ± 36.90 | 5.03 ± 25.18 |
| Ginkgo | 4.0 | 195.83 ± 55.89 | 189.83 ± 92.59 | 182.17 ± 78.42 | 146.33 ± 48.13 |
| Biloba | | | | | |
| extract | | | | | |
| % | | | −6.98 ± 20.97 | −8.66 ± 30.77 | −24.36 ± 22.46 |
| Fucoidan | 4.0 | 186.00 ± 53.94 | 167.50 ± 56.01 | 157.33 ± 79.98 | 156.17 ± 68.02 |
| % | | | −10.98 ± 10.15 | −17.65 ± 27.06 | −17.19 ± 21.39 |
| Fucoidan | 16.0 | 174.67 ± 49.85 | 147.50 ± 57.42 | 131.33 ± 59.03 | 108.83 ± 47.60* |
| % | | | −16.63 ± 21.55 | −25.65 ± 26.24 | −37.76 ± 21.69# |

| | Dosage | After administration (min) | | | | | |
|---|---|---|---|---|---|---|---|
| Groups | (mg/kg) | 15 | 20 | 30 | 45 | 60 | 90 |
| Control | — | 180.17 ± 58.45 | 173.67 ± 51.72 | 172.50 ± 52.51 | 160.67 ± 35.97 | 175.00 ± 55.68 | 171.67 ± 75.79 |
| group | | | | | | | |
| % | | 1.14 ± 31.98 | −0.82 ± 36.50 | −1.02 ± 34.45 | −7.21 ± 29.09 | 2.40 ± 48.24 | −0.90 ± 46.91 |
| Ginkgo | 4.0 | 115.67 ± 23.31* | 113.17 ± 15.74* | 97.50 ± 23.17** | 85.00 ± 15.88*** | 89.67 ± 29.06** | 90.67 ± 23.24* |
| Biloba | | | | | | | |
| extract | | | | | | | |
| % | | −36.33 ± 20.69# | −39.56 ± 12.95# | −47.91 ± 14.66# | −54.15 ± 12.07## | −53.33 ± 11.82# | −50.98 ± 15.48# |
| Fucoidan | 4.0 | 152.33 ± 56.38 | 147.17 ± 40.33 | 141.33 ± 46.11 | 135.00 ± 44.34 | 122.33 ± 34.43 | 135.67 ± 41.34 |
| % | | −18.48 ± 17.07 | −20.39 ± 6.33 | −24.16 ± 10.88 | −27.66 ± 10.78 | −33.27 ± 10.06 | −26.81 ± 9.75 |
| Fucoidan | 16.0 | 112.00 ± 30.66* | 111.00 ± 35.56* | 101.33 ± 49.02* | 108.00 ± 42.69* | 106.67 ± 50.27* | 92.83 ± 40.71* |
| % | | −34.14 ± 13.10# | −35.61 ± 13.28 | −39.86 ± 27.26 | −34.69 ± 29.89 | −38.29 ± 23.37 | −48.20 ± 16.73# |

| | Dosage | After administration (min) | | | | | |
|---|---|---|---|---|---|---|---|
| Groups | (mg/kg) | 120 | 150 | 180 | 240 | 300 | 360 |
| Control | — | 170.83 ± 60.97 | 163.50 ± 49.94 | 160.67 ± 48.39 | 171.33 ± 70.57 | 166.17 ± 64.44 | 164.83 ± 57.85 |
| group | | | | | | | |
| % | | −1.75 ± 41.95 | −5.29 ± 39.29 | −5.46 ± 42.57 | −0.37 ± 47.76 | −1.24 ± 49.80 | −3.96 ± 40.74 |
| Ginkgo | 4.0 | 78.50 ± 17.06** | 79.33 ± 20.61** | 94.00 ± 34.05* | 97.17 ± 36.90* | 100.33 ± 34.48 | 103.33 ± 37.66 |
| Biloba | | | | | | | |
| extract | | | | | | | |
| % | | −56.13 ± 19.53# | −54.67 ± 23.30# | −45.77 ± 32.33 | −43.64 ± 36.39 | −44.08 ± 31.33 | −41.78 ± 35.48 |
| Fucoidan | 4.0 | 133.83 ± 32.90 | 136.67 ± 27.35 | 130.67 ± 36.36 | 139.17 ± 39.75 | 126.50 ± 39.92 | 144.50 ± 49.33 |
| % | | −26.88 ± 8.07 | −24.38 ± 10.91 | −29.13 ± 10.12 | −22.04 ± 27.34 | −31.40 ± 15.10 | −23.22 ± 11.74 |
| Fucoidan | 16.0 | 97.17 ± 38.84* | 92.17 ± 30.97 | 94.67 ± 27.08* | 96.50 ± 40.11* | 110.17 ± 45.58 | 111.17 ± 48.35 |
| % | | −43.84 ± 14.74# | −45.84 ± 13.83# | −42.39 ± 22.30 | −46.86 ± 9.73# | −37.53 ± 19.14 | −37.49 ± 21.21 |

Note:
Compared with the normal salt group,
*p < 0.05
**p < 0.01
***p < 0.001;
compared with the change rate of the normal salt group,
#p < 0.05,
##p < 0.01

TABLE 2
Effect of kelp fucoidan on the range of ischemia in dogs with myocardial
infarction (N-ST, points)(X ± s, n = 6)

| | |
|---|---|
| Dosage | After administration (min) |

| Groups | (mg/kg) | Ligation | 3 | 5 | 10 |
|---|---|---|---|---|---|
| Control | — | 21.50 ± 2.25 | 21.50 ± 3.21 | 20.83 ± 3.25 | 20.17 ± 3.13 |
| group | | | | | |
| % | | | −0.13 ± 3.27 | −3.24 ± 5.10 | −5.89 ± 9.70 |
| Ginkgo | 4.0 | 19.50 ± 2.17 | 19.50 ± 2.07 | 16.50 ± 4.14 | 16.50 ± 2.88 |
| Biloba | | | | | |
| extract | | | | | |
| % | | | 0.55 ± 11.97 | −15.77 ± 17.84 | −14.44 ± 18.47 |
| Fucoidan | 4.0 | 19.83 ± 3.66 | 19.50 ± 3.83 | 18.50 ± 3.56 | 18.00 ± 4.90 |
| % | | | −1.39 ± 13.37 | −5.42 ± 18.92 | −8.95 ± 21.19 |
| Fucoidan | 16.0 | 19.50 ± 2.17 | 18.17 ± 2.71 | 16.67 ± 3.56 | 13.67 ± 4.27* |
| % | | | −6.92 ± 9.56 | −14.94 ± 14.27 | −29.35 ± 21.80# |

| | Dosage | After administration (min) | | | | | |
|---|---|---|---|---|---|---|---|
| Groups | (mg/kg) | 15 | 20 | 30 | 45 | 60 | 90 |
| Control | — | 20.17 ± 2.86 | 19.83 ± 2.64 | 20.83 ± 2.64 | 19.83 ± 3.97 | 21.33 ± 2.25 | 20.83 ± 1.94 |
| group | | | | | | | |
| % | | −5.59 ± 12.07 | −7.47 ± 6.52 | −2.91 ± 4.62 | −8.13 ± 11.17 | 0.33 ± 14.61 | −1.39 ± 18.83 |
| Ginkgo | 4.0 | 16.67 ± 3.27 | 15.83 ± 2.48* | 15.17 ± 2.71** | 12.83 ± 3.55** | 12.83 ± 3.60*** | 12.67 ± 3.83*** |
| Biloba | | | | | | | |
| extract | | | | | | | |
| % | | −13.39 ± 22.25 | −17.63 ± 18.48 | −21.21 ± 16.75# | −34.13 ± 17.12# | −33.53 ± 19.03## | −35.08 ± 18.85# |
| Fucoidan | 4.0 | 19.83 ± 3.76 | 19.17 ± 3.60 | 17.83 ± 2.93 | 17.00 ± 2.10 | 16.00 ± 2.83** | 17.17 ± 3.37* |
| % | | 1.29 ± 17.80 | −2.44 ± 16.48 | −8.36 ± 18.26 | −13.76 ± 9.85 | −18.14 ± 13.25# | −12.36 ± 15.19 |
| Fucoidan | 16.0 | 14.00 ± 1.55*** | 13.33 ± 2.07*** | 14.00 ± 2.61** | 14.17 ± 2.04* | 16.17 ± 3.06** | 15.67 ± 2.88** |
| % | | −27.20 ± 13.32# | −30.71 ± 14.62## | −27.71 ± 14.38## | −27.33 ± 7.29## | −16.45 ± 16.29 | −19.85 ± 11.58 |

| | Dosage | After administration (min) | | | | | |
|---|---|---|---|---|---|---|---|
| Groups | (mg/kg) | 120 | 150 | 180 | 240 | 300 | 360 |
| Control | — | 20.00 ± 2.90 | 19.83 ± 2.04 | 19.83 ± 2.79 | 19.33 ± 2.25 | 17.83 ± 2.48 | 18.17 ± 3.19 |
| group | | | | | | | |
| % | | −5.22 ± 21.49 | −6.71 ± 13.25 | −6.71 ± 15.43 | −8.83 ± 14.59 | −15.51 ± 17.04 | −14.35 ± 16.87 |
| Ginkgo | 4.0 | 13.33 ± 3.72** | 13.00 ± 2.90*** | 14.50 ± 2.35** | 15.00 ± 3.10* | 14.33 ± 3.08 | 14.00 ± 4.52 |
| Biloba | | | | | | | |
| extract | | | | | | | |
| % | | −32.12 ± 15.12# | −33.62 ± 10.78## | −25.79 ± 6.94# | −22.89 ± 15.13 | −25.76 ± 19.42 | −26.96 ± 28.35 |
| Fucoidan | 4.0 | 18.50 ± 3.73 | 19.00 ± 4.00 | 18.83 ± 4.36 | 18.33 ± 4.03 | 18.50 ± 4.32 | 18.50 ± 4.68 |
| % | | −6.41 ± 10.32 | −4.33 ± 9.97 | −5.42 ± 12.71 | −7.79 ± 8.65 | −7.24 ± 10.67 | −7.21 ± 13.90 |
| Fucoidan | 16.0 | 15.00 ± 2.10** | 15.17 ± 2.79** | 13.67 ± 3.39** | 14.83 ± 3.25* | 15.33 ± 1.86 | 15.17 ± 2.64 |
| % | | −22.99 ± 8.50 | −21.89 ± 14.01 | −28.74 ± 20.10 | −23.34 ± 17.14 | −20.64 ± 12.48 | −21.96 ± 11.91 |

Note:
Compared with the normal salt group,
*p < 0.05,
**p < 0.01,
***p < 0.001;
compared with the change rate of the normal salt group,
#p < 0.05,
##p < 0.01

TABLE 3
Effect of kelp fucoidan on the myocardial infarct size in dogs
with myocardial infarction (X ± s, n = 6)

| | | | Infarct/Left |
|---|---|---|---|
| | | Infarct/Heart | ventricular |
| Groups | Dosage | (%) | (%) |
| Normal salt | — | 14.75 ± 1.73 | 21.67 ± 2.42 |
| Ginkgo | 4.0 mg/kg | 11.74 ± 1.66* | 17.43 ± 2.28* |
| Biloba | | | |
| extract | | | |
| Fucoidan | 4.0 mg/kg | 12.56 ± 1.46* | 18.56 ± 1.81* |
| Fucoidan | 16.0 mg/kg | 11.84 ± 1.06** | 17.46 ± 2.14** |

Note:
Compared with the normal salt group,
*p < 0.05
**p < 0.01

### Example 5

### Protection Activity of Marine Extracts Against Experimental Cerebral Ischemia

Method

Effect of Marine Extracts on Breathing Time, Breathing Frequency and Brain Water Content in Decapitated Mice

ICR mice (equally divided between male and female) were divided randomly in a blank control group, a positive control group, and three kelp fucoidan sample groups. The molecular weight of fucoidan administered was between 200 kDa and 400 kDa. The three experimental groups were administered fucoidan at a concentration of 200, 100, and 50 mg/kg, respectively. The mice in experimental groups were administrated fucoidan by tail intravenous injection, and the volume dosage was 10 ml/kg. The positive control group was administrated nimodipine (2 mg/kg) by tail intravenous injection. The model group was administrated normal salt. At 15 minutes after administration, the mice were decapitated by a pair of scissors. The mouth breathing time, breathing frequency and brain water content were recorded and compared with other groups.

Measurement of Brain Water Content

Whole brains were collected. After the wet weight was obtained, they were dried in an oven at 100° C. for 24 hours. The average value was taken to calculate brain water content: brain water content (%)=(wet weight−dry weight)×100%. Brain index: brain index=brain wet weight (g)/body weight (g)×100%.

Effect of Marine Extracts on Cerebral Ischemia in Mice with the Common Carotid Artery Ligation and Reperfusion

Experimental grouping: a control group and a model group (respectively administrating an equal volume of normal saline), a positive control group (nimodipine, 2 mg/kg), and three kelp fucoidan sample groups (fucoidan molecular weight between 200 kDa and 400 kDa) with a concentration of 200, 100, and 50 mg/kg respectively, the injection dosage being 10 mL/mg.

Animal Model Setup:

Grouped mice were respectively administered test substance, nimodipine or normal salt by tail intravenous injection. After 15 minutes, the mice were anesthetized with 3.5% chloral hydrate, fixed in the back. The right and left common carotid artery and vagus nerve were exposed, and 4-0 suture was inserted under the bilateral carotid artery. The suture was tightened to block blood flow for 5 minutes. Then the line was loosened to restore blood flow for 10 minutes. The operation was repeated three times, and an ischemia-reperfusion model in mice was established. After the last reperfusion, the mice were decapitated and brain collected. In the control group, only the bilateral carotid arteries were exposed, without a suture being inserted.

Results

Effect of Marine Extracts on Breathing Time, Breathing Frequency, Brain Index and Brain Water Content in Decapitated Mice

Compared with the blank control group, fucoidan in the 200 mg/kg dosage group can significantly prolong the breathing time (p<0.01), and can significantly increase the breathing frequency (p<0.05). The results are shown in Table 4.

TABLE 4
Effect of kelp fucoidan on breathing time and breathing
frequency in decapitated mice

| | | | | Breathing |
|---|---|---|---|---|
| | Dosage | Animal | Breathing | frequency |
| Groups | (mg/kg) | numbers | time(s) | (times) |
| Blank | — | 10 | 15.9 ± 2.6 | 11.9 ± 3.1 |
| control | | | | |
| Nimodipine | 2 | 10 | 23.8 ± 3.2** | 17.0 ± 3.2** |
| Fucoidan | 200 | 10 | 21.6 ± 1.3** | 14.8 ± 2.1* |
| | 100 | 10 | 17.9 ± 3.2 | 14.2 ± 3.0 |
| | 50 | 10 | 16.0 ± 2.4 | 12.9 ± 3.1 |

Note:
Compared with the blank control group,
*p < 0.05
**p < 0.01

Effect of Marine Extracts on Brain Index and Brain Water Content

Compared with the blank control group, fucoidan in the 200 mg/kg dosage group can significantly decrease the brain index and brain water content (p<0.01), which suggests fucoidan can alleviate brain edema after ischemia-reperfusion and reduce intracranial pressure, improve brain microcirculation. The results are shown in Table 5.

TABLE 5
Effect of kelp fucoidan on brain water content in decapitated mice

| | | | | Brain |
|---|---|---|---|---|
| | | | | water |
| | Dosage | Animal | Brain index | content |
| Groups | (mg/kg) | numbers | (%) | (%) |
| Blank | — | 10 | 1.59 ± 0.12 | 80.6 ± 2.4 |
| control | | | | |
| Nimodipine | 2 | 10 | 1.40 ± 0.14** | 75.8 ± 1.0** |
| Fucoidan | 200 | 10 | 1.45 ± 0.11** | 75.4 ± 1.2** |
| | 100 | 10 | 1.58 ± 0.16 | 78.1 ± 0.8* |
| | 50 | 10 | 1.56 ± 0.10 | 79.0 ± 1.5 |

Note:
Compared with the blank control group,
*p < 0.05
**p < 0.01

Under the same experimental conditions, nimodipine (2 mg/kg) can not only extend breathing time in the decapitated mice and increase breathing frequency, but also lower the brain index and brain water content (p<0.01).

Effect of Marine Extracts on Cerebral Ischemia in Mice with the Common Carotid Artery Ligation and Reperfusion

In this embodiment, the LDH level in the model group has significantly increased compared with the control group. The SOD level decreased significantly (p<0.01), which suggests that the ischemic symptoms of the brain cells death have emerged. Nimodipine can promote the generation of SOD to lower the vitality of LDH. Fucoidan (200 mg/kg) can also promote the generation of SOD, to lower the vitality of LDH (p<0.05 or p<0.01). The results are shown in Table 6.

TABLE 6
Effect of kelp fucoidan on LDH and SOD content
in the brain of ischemic mice

| | Dosage | Animal | | |
|---|---|---|---|---|
| Groups | (mg/kg) | numbers | LDH (U/mg) | SOD (U/mg) |
| Blank | — | 10 | 10.3 ± 1.7 | 163.87 ± 13.08 |
| control | | | | |
| Model | — | 10 | 32.1 ± 8.7## | 123.37 ± 12.96## |
| control | | | | |
| Nimodipine | 2 | 10 | 11.3 ± 6.6** | 144.77 ± 21.61* |
| Fucoidan | 200 | 10 | 11.2 ± 13.7** | 139.22 ± 16.18** |
| | 100 | 10 | 30.4 ± 12.5 | 134.05 ± 16.56 |
| | 50 | 10 | 30.5 ± 16.2 | 130.48 ± 18.38 |

This invention is not to be limited to the specific embodiments disclosed herein and modifications for various applications and other embodiments are intended to be included within the scope of the appended claims. While this invention has been described in connection with particular examples thereof, the true scope of the invention should not be so limited since other modifications will become apparent to the skilled practitioner upon a study of the drawings, specification, and following claims.

All publications and patent applications mentioned in this specification are indicative of the level of skill of those skilled in the art to which this invention pertains. All publications and patent applications mentioned in this specification are herein incorporated by reference to the same extent as if each individual publication or patent application mentioned in this specification was specifically and individually indicated to be incorporated by reference.

The invention claimed is:

1. A method for treating an ischemic cardiovascular or cerebrovascular disease comprising administrating to a patient in the need of such treatment a pharmaceutical composition comprising a fucoidan, wherein said ischemic cardiovascular or cerebrovascular disease comprises a coronary heart disease or a stroke.

2. The method of claim 1, wherein said coronary heart disease comprises a symptomless coronary heart disease, angina, cardiac infarction, arrhythmia, or sudden death.

3. The method of claim 1, wherein said stroke comprises cerebral hemorrhage or cerebral infarction.

4. The method of claim 1, wherein the molecular weight of said fucoidan is between 10 kDa and 1000 kDa.

5. The method of claim 4, wherein the molecular weight of said fucoidan is between 50 kDa and 800 kDa.

6. The method of claim 5, wherein the molecular weight of said fucoidan is between 100 kDa and 700 kDa.

7. The method of claim 6, wherein the molecular weight of said fucoidan is between 150 kDa and 500 kDa.

8. The method of claim 7, wherein the molecular weight of said fucoidan is between 200 kDa and 400 kDa.

9. The method of claim 1, wherein said fucoidan has been extracted from kelp.

10. The method of claim 1, wherein said fucoidan is administered by injection, orally, locally, or intranasally.
